(19) United States
(12) Patent Application Publication
(10) Pub. No.: US 2025/0271407 A1
(43) Pub. Date: Aug. 28, 2025

(54) GAS SENSOR ADAPTER FOR A HIGH FLOW VENTILATION SYSTEM

(71) Applicant: FORM ENERGY, INC., Somerville, MA (US)

(72) Inventor: David RODRIGUEZ, Rocklin, CA (US)

(21) Appl. No.: 19/060,103

(22) Filed: Feb. 21, 2025

Related U.S. Application Data
(60) Provisional application No. 63/557,083, filed on Feb. 23, 2024.

(51) Int. Cl. G01N 33/00; H01M 12/06; H01M 50/358

(52) U.S. Cl. CPC G01N 33/0022; H01M 12/06; H01M 50/358

(57) ABSTRACT

The present disclosure is generally directed to ventilation systems and an assembly for sensing gas concentration in a ventilation system. The assembly includes a body defining an opening and a passage. The assembly includes a first tube supported on the body, the first tube defining a first channel and one or more first apertures, the one or more first apertures in fluid communication with the passage via the first channel. The assembly includes a second tube supported on the body, the second tube defining a second channel and one or more second apertures, the one or more second apertures in fluid communication with the passage via the second channel and, collectively, the one or more first apertures, the first channel, the passage, the second channel, and the one or more second apertures defining at least a portion of a flow path.

### CROSS-REFERENCE TO RELATED APPLICATIONS

This application claims the benefit of priority to U.S. Provisional Patent Application 63/557,083 filed Feb. 23, 2024, the entire contents of which are hereby incorporated herein by reference.

### BACKGROUND

Energy storage technologies are becoming increasingly important in electric power grids. For example, energy storage devices may provide smoothing to better match generation and demand on an electric power grid, as may be beneficial to electric power grids across multiple time scales. While energy storage technologies can support timescales from milliseconds to hours, there is a need for increased availability, reliability, and/or resiliency with reduced costs in energy storage systems.

### SUMMARY

According to one aspect, an assembly for sensing gas concentration in a ventilation system may include a body defining an opening and a passage; a first tube supported on the body, the first tube defining a first channel and one or more first apertures, the one or more first apertures in fluid communication with the passage via the first channel; and a second tube supported on the body, the second tube defining a second channel and one or more second apertures, the one or more second apertures in fluid communication with the passage via the second channel and, collectively, the one or more first apertures, the first channel, the passage, the second channel, and the one or more second apertures defining at least a portion of a flow path.

In some implementations, the one or more first apertures may collectively have a first open area, and the one or more second apertures collectively have a second open area less than the first open area. For example, a ratio of the first open area to the second open area may be greater than 2:1 and less than 5:1. As a specific example, the ratio of the first open area to the second open area may be 3:1. In some instances, each of the one or more first apertures and each of the one or more second apertures may have a diameter of 1 mm to 3 mm. For example, the one or more first apertures and each of the one or more second apertures may have a diameter of 2 mm.

In certain implementations, at least a portion of the passage may be threaded such that the passage is releasably connectable in threaded engagement with a gas sensor.

In some implementations, an outer surface of the body may be threaded such that the body is threaded engagement with ducting of the ventilation system. For example, the outer surface of the body may have a ¾ inch National Pipe Tapered (NPT) thread.

In certain implementations, the one or more first apertures, the first channel, the passage, the second channel, and the one or more second apertures may collectively define a flow path in which volumetric air flow rate in the passage, from the first channel to the second channel, is 0.001 percent to 0.01 percent of volumetric air flow rate incident on an external surface of the first tube along the one or more first apertures of the first tube.

In some implementations, the body may define a first through hole and a second through hole, the first tube is supported in the first through hole, and the second tube is supported in the second through hole. The first tube may be brazed or welded in the first through hole. Further, or instead, the second tube may be brazed or welded in the second through hole.

In certain implementations, the first tube may have a first length extending from the body, the second tube has a second length extending from the body, and the first length is equal to the second length.

In some implementations, the first tube may have a first minimum cross-sectional area transverse to a length dimension of the first tube, the second tube has a second minimum cross-sectional area transverse to a length dimension of the second tube, and the first minimum cross-sectional area is equal to the second minimum cross-sectional area.

In certain implementations, at least one of the first tube or the second tube may have a closed end away from the body.

In some implementations, the assembly may further include a gas sensor disposed in the passage via the opening of the body. As an example, the gas sensor may have a rated flow rate greater than 0.5 L/min and less than 5 L/min. In some instances, the gas sensor may be a hydrogen sensor.

According to another aspect, a ventilation system for electrochemical energy storage and discharge, may include: ducting defining a shared vent connectable in fluid communication with a plurality of electrochemical cells; at least one fan in fluid communication with the shared vent; an adapter including a body, a first tube, and a second tube, the body defining an opening and a passage in fluid communication with one another, the body mechanically coupled to the ducting with the first tube and the second tube in the shared vent; and a gas sensor in fluid communication with the passage, and the passage in fluid communication with the shared vent via the first tube and the second tube.

In certain implementations, the first tube may define one or more first apertures, the second tube defines one or more second apertures, and the one or more first apertures and the one or more second apertures are disposed on an inlet side of the at least one fan.

In some implementations, the body of the gas sensor may be in threaded engagement with the ducting.

In certain implementations, the gas sensor may be in threaded engagement with the body of the adapter.

In some implementations, the gas sensor may have a rated volumetric flow rate less than 5 L/min, and the at least one fan has a volumetric flow rate is 28.3 kL/min to 566 kL/min.

In certain implementations, volumetric flow rate at the gas sensor may be less than 5/L/min at a constant absolute pressure of 1.0±0.2 atm.

In some implementations, the gas sensor may be a hydrogen sensor.

### DESCRIPTION OF THE DRAWINGS

FIG. 1A is a perspective a system for gas management of metal-air batteries, with the system including a plurality of electrochemical cells and a manifold, the system shown without an enclosure and hardware of the enclosure, and gas flow from the plurality of electrochemical cells and the manifold indicated by arrows.

FIG. 1B is a schematic representation of gas flow through the system of FIG. 1A, with representation of the system and associated gas flow of FIG. 1A simplified for the sake of clarity and the system shown with an enclosure.

FIG. 1C is a schematic representation of a thermal management system of the system of FIG. 1A, with representation of air flow for thermal management of the system of FIG. 1A simplified for the sake of clarity, the system shown with an enclosure, and the gas management system not shown for the sake of clarity.

FIG. 2 is a flowchart of an exemplary method of gas management of metal-air batteries.

FIG. 3A is a perspective view of an assembly for sensing gas concentration in the system of gas management of FIG. 1A, the assembly including an adapter and a gas sensor, and the assembly shown mechanically coupled to a portion of ducting of the system of gas management of FIG. 1A.

FIG. 3B is a perspective, partially exploded view of the assembly of FIG. 3A, shown with the gas sensor separated from the adapter.

FIG. 3C is a perspective view of the adapter of the assembly of FIG. 3A.

FIG. 3D is a cross-section of the perspective view of the adapter of the assembly of FIG. 3A, the cross-section along 3D-3D in FIG. 3C.

FIG. 3E is a top view of the adapter of the assembly of FIG. 3A.

FIG. 3F is a left side view of the adapter of the assembly of FIG. 3A.

FIG. 3G is a right side view of the adapter of the assembly of FIG. 3A.

FIG. 3H is a close-up view of the area of detail 3H in FIG. 3F.

FIG. 3I is a close-up view of the area of detail 3I in FIG. 3G.

FIG. 3J is a bottom view of a body of the adapter of the assembly of FIG. 3A.

FIG. 4 is a cross-sectional view of the adapter of the assembly of FIG. 3A with schematic representation of a flow path of gas from a shared vent of the gas management of FIG. 1A through the adapter of the assembly of FIG. 3A for measurement by the gas sensor of the assembly of FIG. 3A.

Like reference symbols in the various drawings indicate like elements.

### DETAILED DESCRIPTION

Embodiments will be described in detail with reference to the accompanying drawings, in which exemplary embodiments are shown. The foregoing may, however, be embodied in many different forms and should not be construed as limited to the exemplary embodiments set forth herein.

The various embodiments of systems, equipment, techniques, methods, activities and operations set forth in this specification may be used for various other activities and in other fields in addition to those set forth herein. Additionally, these embodiments, for example, may be used with: other equipment or activities that may be developed in the future; and, with existing equipment or activities which may be modified, in-part, based on the teachings of this specification. Further, the various embodiments and examples set forth in this specification may be used with each other, in whole or in part, and in different and various combinations. Thus, the configurations provided in the various embodiments of this specification may be used with each other. For example, the components of an embodiment having A, A′ and B and the components of an embodiment having A″, C and D can be used with each other in various combinations, e.g., A, C, D, and A. A″ C and D, etc., in accordance with the teaching of this Specification.

For the sake of clear and efficient description, elements with numbers having the same last two digits in the disclosure that follows shall be understood to be analogous to or interchangeable with one another, unless otherwise specified or made clear from the context, and, therefore, are not described separately from one another, except to note differences and/or to emphasize certain features.

The present disclosure is directed to systems, methods, and devices for electrochemical energy storage systems, such as metal-air battery systems. Various embodiments may be applicable to gas management in electrochemical energy storage systems, such as metal-air battery systems. Various embodiments may be applicable to explosive and/or flammable gas management in electrochemical energy storage systems, such as metal-air battery systems.

Various embodiments may provide devices and/or methods for use in long-duration, and ultra-long-duration, low-cost, energy storage. Herein, “long duration” and “ultra-long duration” and similar such terms, unless expressly stated otherwise, shall be understood to include periods of energy storage of 8 hours or longer, such as periods of energy storage of 8 hours, periods of energy storage ranging from 8 hours to 20 hours, periods of energy storage of 20 hours, periods of energy storage ranging from 20 hours to 24 hours, periods of energy storage of 24 hours, periods of energy storage ranging from 24 hours to a week, periods of energy storage ranging from a week to a year (e.g., such as from several days to several weeks to several months), etc. and would include long duration energy storage (LODES) systems. Further, the terms “long duration” and “ultra-long duration”, “energy storage cells” including “electrochemical cells”, and similar such terms, unless expressly stated otherwise, shall be understood to include electrochemical cells that may store energy over time spans of days, weeks, or seasons. As used herein, unless a contrary intention is explicitly stated or made clear from the context, the term “duration” shall be understood to refer to a ratio of energy to power of an energy storage system. For example, a system with a rated energy of 24 MWh and a rated power of 8 MW has a duration of 3 hours, and a system with a rated energy of 24 MWh and a rated power of 1 MW has a duration of 24 hours. Physically, the duration may be interpreted as the run-time of the energy storage system at maximum power.

In general, a long duration energy storage cell may be a long duration electrochemical cell. Such a long duration electrochemical cell may store electricity generated from an electrical generation system, when: (i) the power source or fuel for the electrical generation system is available, abundant, inexpensive, or otherwise advantageous; (ii) the power requirements or electrical needs of the electrical grid, customer or other user, are less than the amount of electricity generated by the electrical generation system, the price paid for providing such power to the grid, customer or other user, is below an economically efficient point for the generation of such power (e.g., cost of generation exceeds market price for the electricity), and combinations and variations of these; and (iii) combinations and variations of (i) and (ii) as well as other reasons. The electricity stored in the long duration electrochemical cell can then be distributed to the grid, customer or other user, at times when it is economical or otherwise needed. For example, the electrochemical cells may be configured to store energy generated by solar cells during the summer months, when sunshine is plentiful and solar power generation exceeds power grid requirements. Continuing with this example, the electrochemical cells may discharge the stored energy during the winter months, when sunshine may be insufficient for energy generated by the solar cells to satisfy power grid requirements.

Various embodiments may provide devices and/or methods for use in bulk energy storage systems, such as long duration energy storage (LODES) systems, short duration energy storage (SDES) systems, etc. As an example, various embodiments may provide configurations and controls for batteries of bulk energy storage systems, such as batteries for LODES systems.

U.S. Pat. App. Pub. 2021/0028457, entitled “LOW COST METAL ELECTRODES,” which published on Jan. 28, 2021, the entire contents of which are hereby incorporated herein by reference, describes various aspects of electrochemical cells, such as rechargeable batteries using metal electrodes (e.g., iron negative electrodes), and design, manufacture, and processing features of electrochemical cells, such as rechargeable batteries using iron metal electrodes (e.g., iron negative electrodes), with which various embodiments described herein may be used and into which various embodiments described herein may be incorporated. Additionally, U.S. Pat. App. Pub. 2021/0028457 provides examples of metal materials (e.g., iron materials) with which various embodiments described herein may be used. Further, U.S. Pat. App. Pub. 2021/0028457 describes bulk energy storage systems, such as LODES systems, with which various embodiments described herein may be used and into which various embodiments as described herein may be incorporated.

As used herein, a “module” may include a string of unit electrochemical cells (e.g., a string of batteries). Multiple modules (or multiple units or cells) may be connected together to form battery strings.

Attention is now directed to gas management of oxyhydrogen mixtures that are exhausted from the electrochemical cells of the modules.

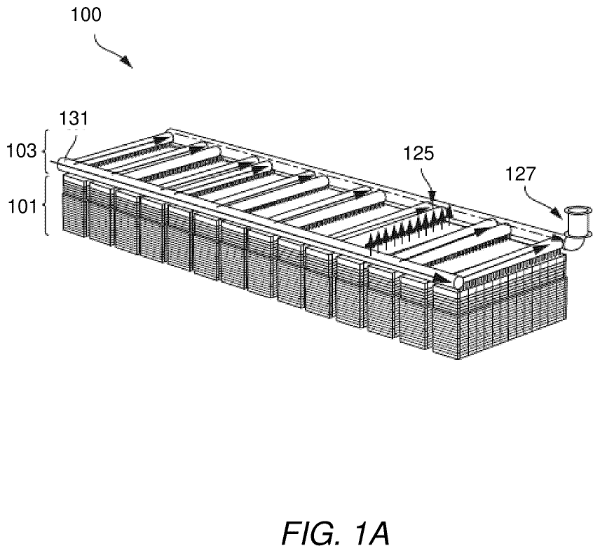

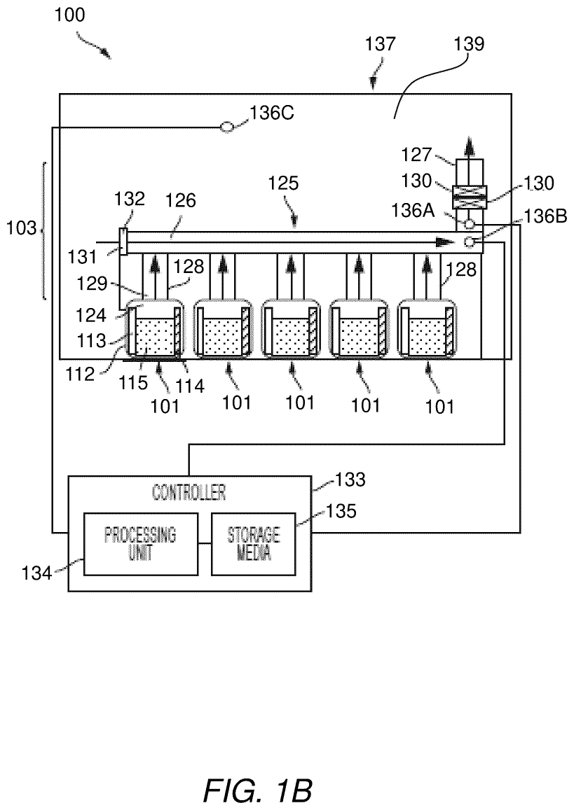

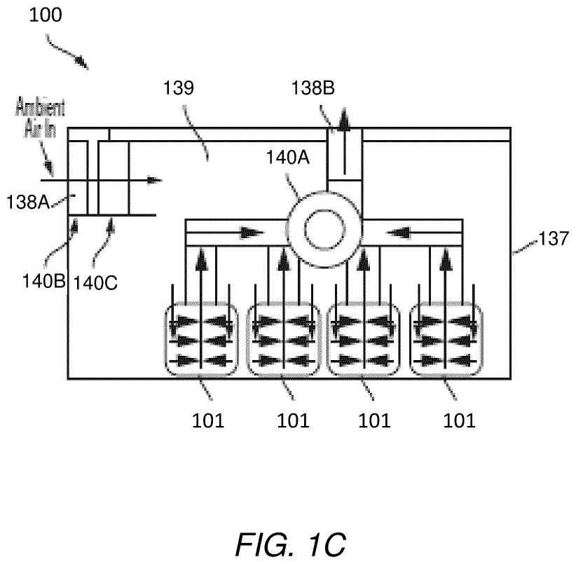

Referring now to FIGS. 1A and 1B, a system 100 for gas management of metal-air batteries may include a plurality of electrochemical cells 101 and a manifold 103. Each one of the plurality of electrochemical cells 101 may include at least one air electrode 113, a metal electrode 114, a vessel 112, and a liquid electrolyte 115 between the at least one air electrode 113 and the metal electrode 114 in the vessel 112. Thus, for example, the plurality of electrochemical cells 101 may include iron-air type battery cells, zinc-air type battery cells, lithium-air battery cells, or a combination thereof.

Each one of the plurality of electrochemical cells 101 may define a respective headspace 124 above the liquid electrolyte 115 in the vessel 112. The manifold 103 may include ducting 125 defining a shared vent 126 and an outlet region 127. The respective headspace 124 of each one of the plurality of electrochemical cells 101 may be fluidically coupled to the shared vent 126 and in fluid communication with the outlet region 127 of the ducting 125 such that the plurality of electrochemical cells 101 may vent to the shared vent 126, where oxyhydrogen in the shared vent 126 may be diluted by ambient air in the shared vent 126.

As compared to sweeping oxyhydrogen from the respective headspace of each of a plurality of electrochemical cells, venting oxyhydrogen from the plurality of electrochemical cells 101 into the shared vent 126 of the ducting 125 may significantly reduce complexity and/or cost associated with gas management through, among other things, less competition for air used for thermal management of the plurality of electrochemical cells 101. Further, or instead, as also compared to sweeping oxyhydrogen from the respective headspace of each of a plurality of electrochemical cells, venting oxyhydrogen from the plurality of electrochemical cells 101 into the shared vent 126 for dilution in the shared vent 126 may reduce the amount of electrolyte mist (e.g., KOH mist) entrained in gas exhausted from the system 100 to an ambient environment.

In certain implementations, the system 100 for gas management may further or instead include a plurality of risers 128 each defining an instance of a cell vent 129. The headspace 124 of each one of the plurality of electrochemical cells 101 may be fluidically coupled to the shared vent 126 via at least one instance of the cell vent 129 of the plurality of risers 128. The plurality of risers 128 may provide a flow restriction and/or a non-linear flow path from the respective headspace 124 to the shared vent 126 as may be useful for reducing the likelihood of unwanted liquid (e.g., electrolyte mist) from moving into the shared vent 126 from the respective headspace 124. While each respective headspace 124 may exhaust to a single instance of one of the plurality of risers 128, it shall be appreciated that the respective headspace 124 of each of one of the plurality of electrochemical cells 101 may exhaust to the shared vent 126 via more than one instance of the plurality of risers 128.

In some instances, the system 100 may include at least one instance of a fan 130 in fluid communication with the shared vent 126, with the fan 130 operable to move gas along the shared vent 126 and out of the ducting 125 via the outlet region 127. It shall be appreciated that operation of the fan 130 to move gas along the shared vent 126 may reduce the amount of time that that oxyhydrogen mixture remains in the shared vent 126 before being exhausted to an ambient environment through the outlet region 127. Further, or instead, in instances in which operation of the fan 130 moves air through the shared vent 126, the fan 130 may dilute the oxyhydrogen mixture with air, thus reducing the likelihood of explosion of the oxyhydrogen mixture relative to otherwise identical conditions without dilution of the oxyhydrogen mixture with ambient air.

In general, the fan 130 may be disposed in any one more of various positions relative to the shared vent 126 with the fan 130 in fluid communication with the shared vent 126. As an example, the fan 130 may be disposed in the shared vent 126, as may be useful for using the ducting 125 to provide protection and support for the fan 130. Additionally, or alternatively, the fan 130 disposed in the shared vent 126 may facilitate making efficient use of power provided to the fan 130 to move gas through the shared vent 126. In some examples, the fan 130 may be supported by the ducting 125 at the outlet region 127 defined by the ducting 125, as may be useful for providing access to the fan 130 for repair/replacement while making efficient use of power to the fan 130 to move gas through the shared vent 126.

In certain implementations, the fan 130 may be oriented relative to the shared vent 126 such that the fan 130 is operable to form negative pressure in the shared vent 126 relative to ambient air pressure at the outlet region 127 of the ducting 125. As compared to positive pressure in the shared vent 126, the fan 130 operable to form negative pressure in the shared vent 126 may be useful for reducing the likelihood of the oxyhydrogen mixture inadvertently (e.g., via a leak) moving out of the shared vent 126. That is, the fan 130 operable to form negative pressure in the shared vent 126 may reduce the likelihood that an unsafe concentration of hydrogen may form outside of the shared vent 126. Further, or instead, given that the fan 130 operable to form negative pressure in the shared vent 126 pulls gas from the shared vent 126 to exhaust the gas to an ambient environment via the outlet region 127, the fan 130 may be explosion-proof rated. In the context of the fan 130, the term “explosion-proof” shall be understood to include any fan having a housing that contains any explosion originating from within the housing and prevents and sparks from exiting the housing. Thus, it shall be appreciated instances in which the fan 130 has an explosion-proof rating may reduce the likelihood that the fan 130 itself may serve as an ignition source for an ignitable mixture of oxyhydrogen (or other ignitable mixture) moving through the fan 130 with the fan 130 in operation.

In some implementations, the system 100 may include a plurality of instances of the fan 130 to provide backup redundancy in the system 100. As an example, the system 100 may include a first instance and a second instance of the fan 130, which is useful for reducing the likelihood of an accumulation of a high concentration in the shared vent 126 in the event of failure of one instance of the fan 130. Continuing with this example, the first instance of the fan 130 and the second instance of the fan 130 may be powered separately from one another (e.g., connected to independent power sources) such that failure of a given power source does not act as a single point of failure in operation of the system 100. That is, in the event of a failure of the power source of the first instance of the fan 130, the second instance of the fan 130 may continue to operate with power from a separate power source. Further, or instead, only one of the first instance or the second instance of the fan 130 may be operable at a time, as may be useful for achieving redundancy while making efficient use of power required for gas management by the system 100.

In certain implementations, the ducting 125 may define an inlet region 131, and the fan 130 may be operable to move air (e.g., from an ambient environment outside of the ducting 125 and the plurality of electrochemical cells 101) into the shared vent 126 via the inlet region 131. For example, the respective headspace 124 of each one of the plurality of electrochemical cells 101 may be in fluid communication with the shared vent 126 along the ducting 125 between the inlet region 131 and the outlet region 127 and, as the fan 130 moves air into the shared vent 126 via the inlet region 131, the air moving through the shared vent 126 may mix with gas moving into the shared vent 126 from the respective headspace 124 of each one of the plurality of electrochemical cells 101 and the mixture of gas may ultimately exit the shared vent 126 via the outlet region 127. In certain instances, the system 100 may include a filter 132 disposed along the inlet region 131 of the ducting, as may be useful for reducing or eliminating moisture, dust, and/or other debris from entering the shared vent 126 to prematurely degrade components of the system 100.

The system 100 may include a controller 133 operable to determine conditions that are unsafe or have an increased likelihood of becoming unsafe and to take one or more corrective actions as described herein. The controller 133 may include a processing unit 134 and storage media 135 (e.g., non-transitory, computer-readable storage media) in electrical communication with one another. The storage media 135 may have stored thereon computer-executable instructions that, when executed by the processing unit 134 carry out any one or more of the various, different aspects of methods of gas management described herein.

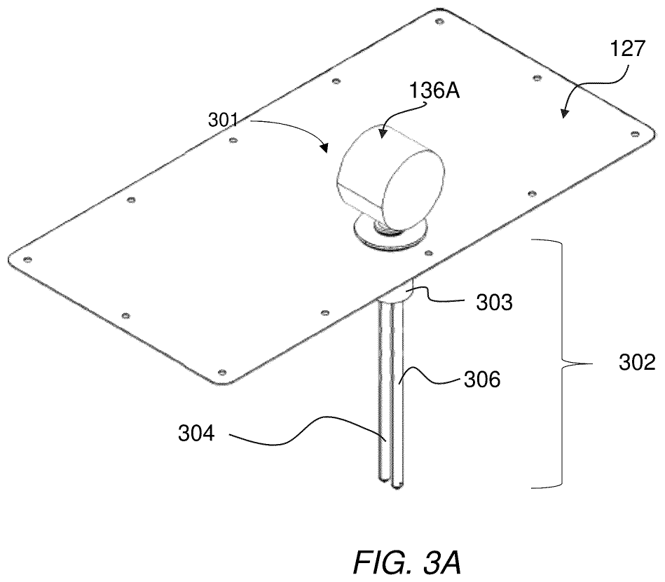

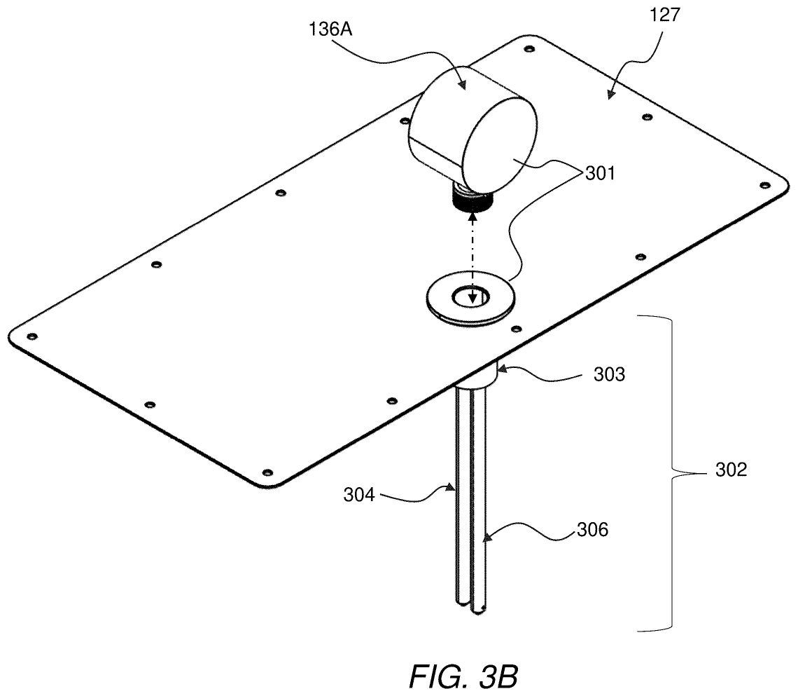

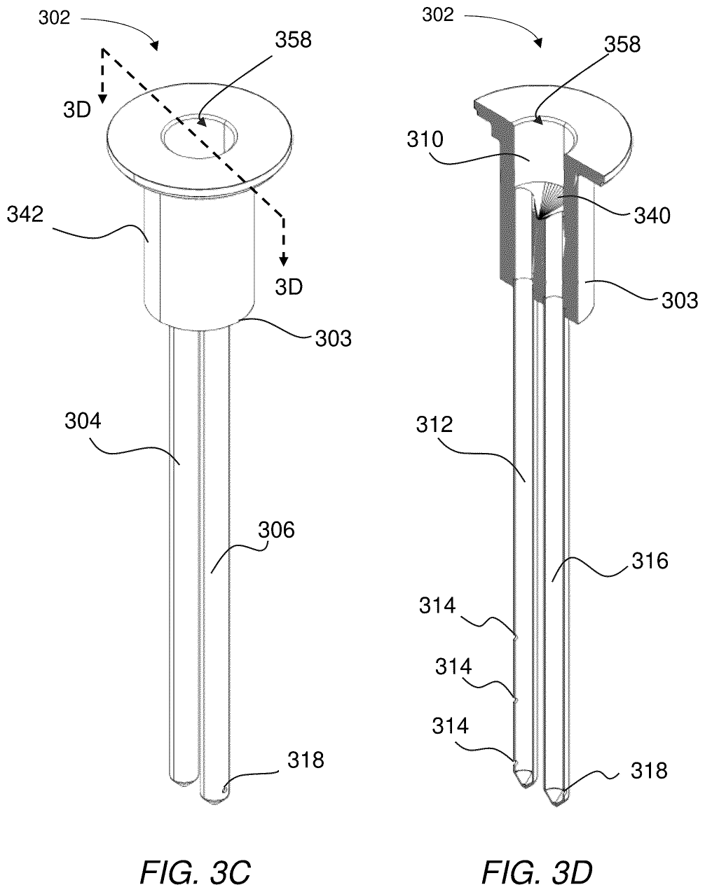

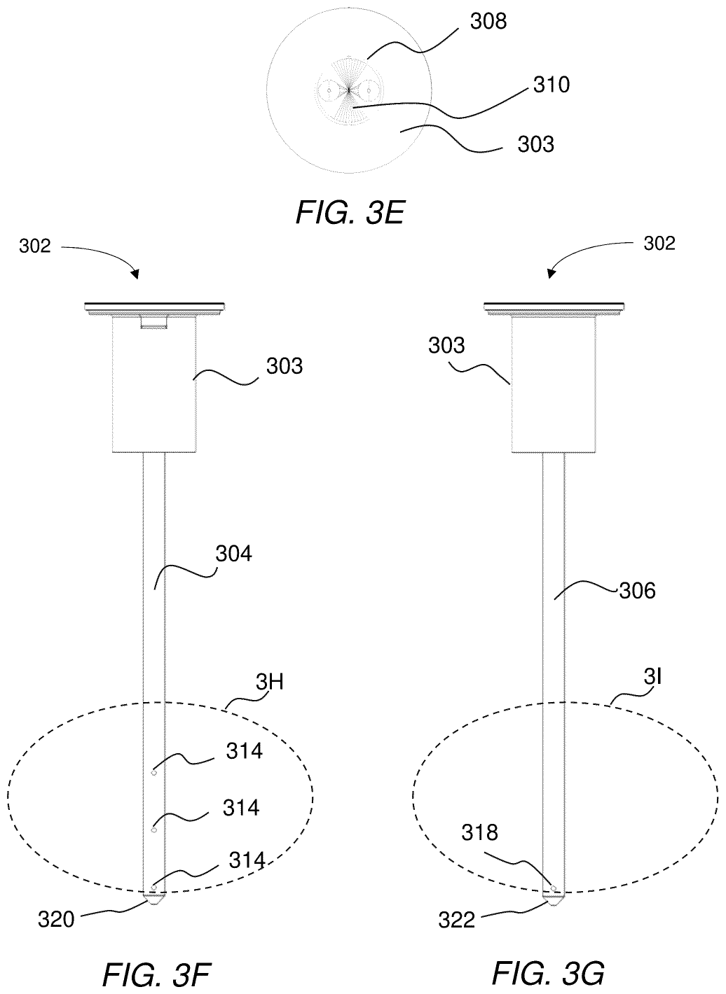

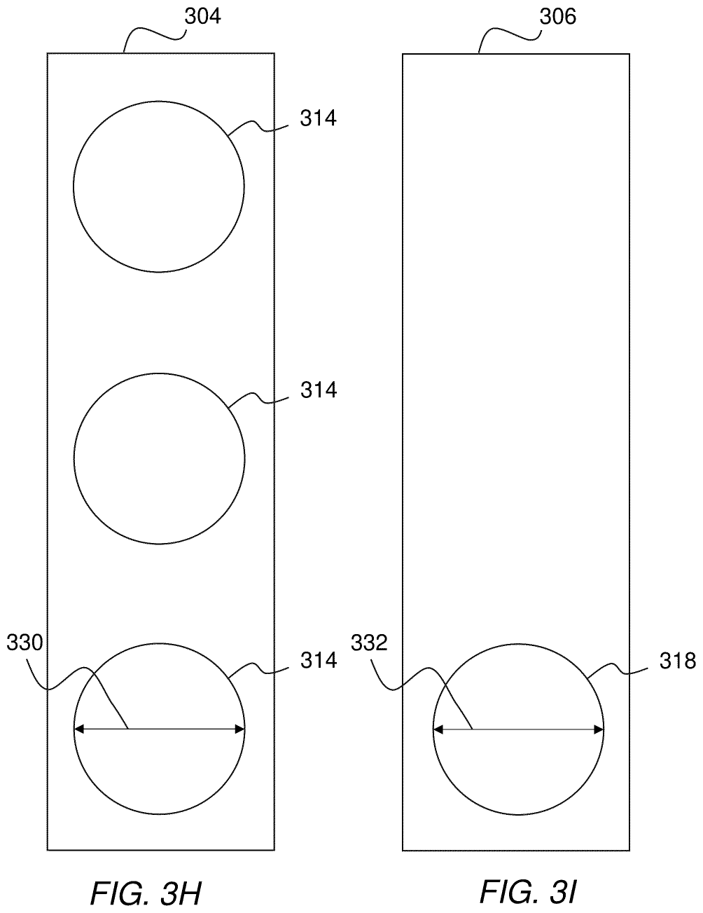

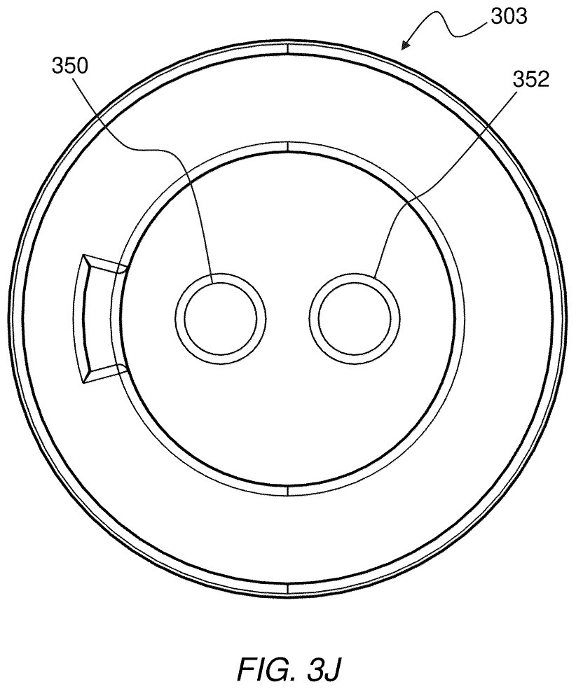

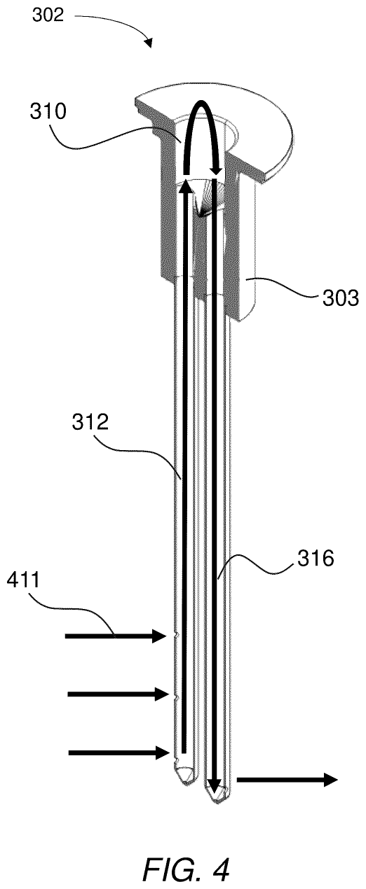

In certain implementations, the system 100 may include a first gas sensor 136A (e.g., any one or more different types of palladium-based hydrogen sensors or other types of hydrogen sensor known in the art) in electrical communication with the controller 133 (e.g., with the processing unit 134 of the controller 133) and in fluid communication with the shared vent 126 to sense hydrogen via an assembly 301 (FIGS. 3A-3I and 4). As also described in greater detail below, the controller 133 may be configured to receive a first signal from the first gas sensor 136A and to control speed of the fan 130 based on the first signal received from the first hydrogen sensor 133A. In certain implementations, the first gas sensor 136A may receive gas sampled from the shared vent 126 between the outlet region 127 of the ducting 125 and the fluidic coupling of the respective headspace 124 of each one of the plurality of electrochemical cells 101 to the shared vent 126 of the ducting 125. Because the first gas sensor 136A receives a gas sample from a position downstream of the respective headspace 124 of each one of the plurality of electrochemical cells 101 in a direction of air movement being forced through the shared vent 126 by the fan 130, the gas moving through the shared vent 126 has collected oxyhydrogen from each one of the plurality of electrochemical cells 101 at this sampling position monitored by the first gas sensor 136A. Thus, this position of the shared vent 126 monitored by the first gas sensor 136A may be more likely to detect a global maximum hydrogen concentration within the shared vent 126, as compared to other hydrogen monitoring positions within the shared vent 126. Stated differently, the position monitored by the first gas sensor 136A may increase the likelihood of detecting excessive levels of hydrogen or another sensed gas in the shared vent 126 if such levels exist in the shared vent 126, while using only a single sensor or a limited number of sensors.

In certain implementations, the system 100 may further include a second gas sensor 136B in electrical communication with the controller 133 (e.g., with the processing unit 134). As described in greater detail below, the controller 133 may be further configured to receive a second signal from the second gas sensor 136B and to control speed of the fan 130 based on the first signal and the second signal. As an example, the first gas sensor 136A and the second gas sensor 136B may be each receive samples of gas (e.g., using respective instances of the assembly 301 in FIG. 3A, described in greater detail below) from the same or nearly the same position in the shared vent 126, as may be useful for providing redundancy in the event that one of the gas sensors fails or loses communication with the controller 133. As another example, the second gas sensor 136B may receive a gas sample from a position in the shared vent 126 away from the measurement made by the first gas sensor 136A, as may be useful for determining certain anomalous conditions resulting in an unusual concentration gradient of the gas being monitored (e.g., hydrogen) in the shared vent 126. In such instances, the controller 133 may initiate one or more corrective actions based on the highest of the first signal and the second signal.

The first gas sensor 136A and/or the second gas sensor 136B may be operable to detect the concentration of hydrogen in the volumetric flow of gas moving through the shared vent 156. For example, at least one of the first gas sensor 136A or the second gas sensor 136B may be a palladium-based sensor, which leverages the ability of palladium to absorb hydrogen, causing a measurable change in its electrical resistance. As hydrogen molecules diffuse into the palladium layer, the formation of a metal hydride may alter the conductivity of the sensor. This change in conductivity may be converted into an electrical signal proportional to hydrogen concentration in the gas to which the sensor is exposed. Alternatively, or in addition, at least one of the first gas sensor 136A or the second gas sensor 136B may operate based on a catalytic reaction in which hydrogen interacts with oxygen on a sensing element, producing heat or an electrochemical reaction that generates a current or voltage signal.

In certain implementations, the system 100 may include an enclosure 137, as may be useful for storing and/or shipping the system 100 with little or no need for specialized equipment and/or assembly at the point of use of the system 100. In particular, the enclosure 137 may define an intake opening 138A, an exhaust opening 138B, and a chamber 139, with the intake opening 138A and the exhaust opening 138B in fluid communication with one another via an environment of the chamber. The plurality of electrochemical cells 101 and the manifold 103 may be disposed in the environment of the chamber 139 with the respective headspace 124 of each one of the plurality of the electrochemical cells 101 and the shared vent 126 of the ducting 125 fluidically isolated from the environment of the chamber 139, and the outlet region 127 of the ducting 125 may be in fluid communication with an ambient environment outside of the enclosure 137. That is, under normal operating conditions, the enclosure 137 protects the plurality of electrochemical cells 101 and the manifold 103 (e.g., from degradation associated with weather) without an accumulation of hydrogen in the environment of the chamber 139.

While the enclosure 137 is useful for packaging the system 100 in a form factor that is amenable to being transported and is additionally, or alternatively, useful for protecting components of the system 100, it shall be appreciated that the environment of the chamber 139 may be associated with certain dangerous or potentially dangerous conditions under anomalous situations in which hydrogen inadvertently collects in the environment of the chamber 139. Accordingly, in some implementations, the system 100 may include a leak sensor 136C (e.g., a hydrogen sensor similar to the first gas sensor 136A and the second gas sensor 136B) arranged to sense concentration of hydrogen in the environment of the chamber 139. Continuing with this example, the controller 133 may be in electrical communication with the leak sensor 136C. The controller 133 may be further configured to receive a third signal from the leak sensor 136C and to take one or more corrective actions based at least in part on the third signal of the leak sensor 136C in instances in which the leak sensor 136C detects high concentrations of hydrogen in the environment of the chamber 139.

Referring now to FIGS. 1A-1C, in certain implementations, a corrective action initiated by the controller 133 based on the third signal from the leak sensor 136C may include controlling one or more thermal management components operable to keep the plurality of electrochemical cells 101 under normal operating conditions. For example, the system 100 may include a cooling fan 140A in fluid communication with the environment of the chamber 139 and activatable to pull air into the environment of the chamber 139 via the intake opening 138A and exhaust air from the environment of the chamber 139 via the exhaust opening 138B. That is, while the cooling fan 140A may ordinarily cool the plurality of electrochemical cells 101 under normal operating conditions, the air change resulting from operation of the cooling fan 140A may advantageously remove hydrogen from the environment of the chamber 139 when high concentrations of hydrogen are detected by the leak sensor 136C or under other anomalous conditions (e.g., loss of communication between the leak sensor 136C and the controller 133). As an example, with the cooling fan 140A actuated and operating at a maximum rated speed, the air change of the environment of the chamber 139 may be less than about 30 seconds, as may be useful for quickly lowering hydrogen concentration in the environment of the chamber 139 to restore safe operating conditions.

In certain implementations, the system 100 may include a filter material 140B supported along the intake opening 138A of the enclosure 137. The filter material 140B may be generally useful for reducing the likelihood of ingress of moisture, debris, or other unwanted material into the environment of the chamber 139 as the cooling fan 140A draws air through the filter material 140B and into the environment of the chamber 139. In the context of gas management, such reduction of unwanted material in the environment of the chamber 139 may be useful for reducing the likelihood of certain failure modes (e.g., failure of the cooling fan 140A, failure of communication between the leak sensor 136C and the controller 133, etc.) that may serve as a basis for initiating one or more corrective actions up to and including interruption of operation of the system 100.

In some implementations, the system 100 may include evaporative media 140C supported along the intake opening 138A of the enclosure 137. Thus, as air moves through the intake opening 138A of the enclosure 137, the evaporative media 140C may evaporate and, in doing so, cool the air entering the environment of the chamber 139. While such cooling is primarily associated with thermal management of the plurality of electrochemical cells 101 under normal operating conditions, it shall be appreciated that the cooling provided by the evaporative media 140C may reduce temperature in the environment of the chamber 139, thus lowering the likelihood of ignition of a hydrogen-containing gas mixture in the chamber 139 as compared to the likelihood of ignition at higher temperatures that would otherwise occur in the absence of the evaporative media 140C.

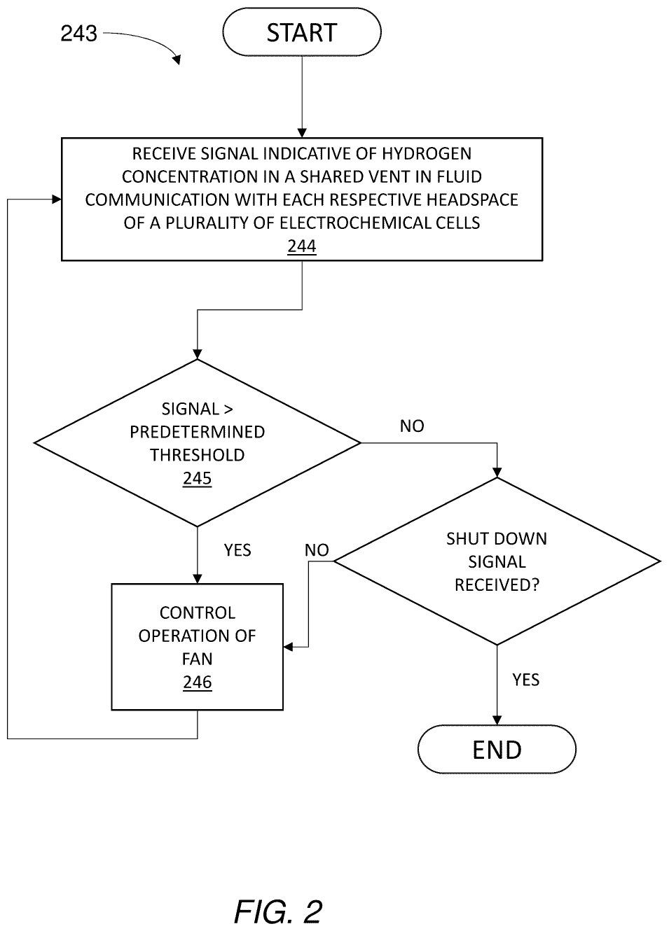

FIG. 2 is a flowchart of an exemplary method 243 of gas management of metal-air batteries. Unless otherwise specified or made clear from the context, it shall be understood that any one or more of various different aspects of the exemplary method 243 may be carried out by the controller 133 (FIG. 1B) in electrical communication with one or more of various different sensors, fans, and/or other components described herein. For example, the storage media 135 (FIG. 1B) may have stored thereon instructions for causing the processing unit 134 (FIG. 1B) to carry out one or more aspects of the exemplary method 243.

As shown in step 244, the exemplary method 243 may include receiving, from each of one or more hydrogen sensors, a respective signal indicative of hydrogen concentration in a shared vent defined by ducting and in fluid communication between each respective headspace of a plurality of electrochemical cells and an outlet defined by the ducting. As an example, the respective signal from at least one of the one or more hydrogen sensors may be indicative of hydrogen concentration in the shared vent upstream of the at least one fan relative to a direction of gas flow through the at least one fan toward the outlet of the ducting. Further, or instead, the respective signal from the at least one of the one or more hydrogen sensors is indicative of hydrogen concentration in the shared vent downstream of fluidic coupling of the headspaces of the electrochemical cells to the shared vent relative to the direction of gas flow through the at least one fan toward the outlet of the ducting.

In certain implementations, receiving the respective signal from the one or more hydrogen sensors may include determining whether each of the one or more hydrogen sensor is operational. For example, the absence of a signal received from a given one of the one or more hydrogen sensors at a time when such a signal may be indicative that the given hydrogen sensor has stopped working and/or has lost communication with the controller. If the sensor is determined to be non-operational, one or more corrective actions may be initiated to reduce the likelihood of an anomalous condition developing while the given hydrogen sensor is non-operational. As an example, at least one fan used to move air through the shared vent of the ducting may be controlled to operate at 100 percent (or another predetermined percentage) of rated operating speed such that a high volumetric flow rate of air may continue to move through the shared vent. While such operation of the fan is generally inefficient under normal operating conditions, it shall be appreciated that operating the fan at high speed in this context is generally done for a short period of time (e.g., until a corrective action can be taken with respect to the non-operational hydrogen sensor.

As shown in step 245, the exemplary method 243 may include comparing the respective signal from each of the one or more hydrogen sensors to at least one predetermined threshold. As an example, the at least one predetermined threshold may correspond to a hydrogen concentration less than the lower flammability limit of hydrogen in air at a predetermined temperature and a predetermined pressure (e.g., at 25° C. at atmospheric pressure). While the at least one predetermined threshold may be a single threshold in some instances, it shall be appreciated that the at least one predetermined threshold may include a first predetermined threshold and a second predetermined threshold, and the second predetermined threshold may be greater than the first predetermined threshold.

As an example, the first predetermined threshold and the second predetermined threshold may correspond to a window of hydrogen concentration below the lower flammability limit of hydrogen at the predetermined temperature and the predetermined pressure such that the window may form the basis of closed-loop control of any one or more of the various different components of gas management systems described herein. As an example, the first predetermined threshold may correspond to 12.5% of the lower flammability limit of hydrogen in air at a predetermined temperature and a predetermined temperature while the second predetermined threshold may correspond to 25% of the lower flammability limit of hydrogen in air at the same conditions. These values provide a useful margin of safety relative to the lower flammability limit such that one or more corrective actions may be taken prophylactically-namely, before hydrogen concentration within the shared vent becomes dangerous.

As shown in step 246, the exemplary method 243 may include, based on comparison of the respective signal of each of the one or more hydrogen sensors to the at least one predetermined threshold in step 245, controlling at least one fan in fluid communication with the shared vent and operable to move gas along the shared vent and out of the ducting via the outlet. For example, controlling the at least one fan may include forming vacuum pressure in the shared duct, as may be useful for reducing the likelihood that hydrogen in the shared duct may leak out of the ducting.

Returning to the example in which the at least one predetermined threshold includes a first predetermined threshold and the second predetermined threshold, controlling that at least one fan may include adjusting an operating speed of the at least one fan if the respective signal from any one of the hydrogen sensors is between the first predetermined threshold and the second predetermined threshold. In some implementations, adjusting the operating speed of the at least one fan may include ramping up (e.g., progressively increasing the speed over time) the operating speed of the at least one fan if the respective signal from any of the one or more hydrogen sensors is between the first predetermined threshold and the second predetermined threshold.

Continuing with this example, once the respective signal from each of the one or more hydrogen sensors indicates that the hydrogen concentration in the shared vent is below the first predetermined threshold, the at least one fan may be shut off to reduce the amount of power consumed for gas management. Continuing still further with this example, signals from the one or more hydrogen sensor indicating that hydrogen concentration in the shared vent is below the first predetermined threshold and the at least one fan shut off, controlling the at least one fan may include periodically activating the at least one fan at one or more predetermined intervals. Such periodic activation of the at least one fan may move any hydrogen from the shared vent and, further or instead, may provide conditions useful for periodically measuring hydrogen concentration to determine whether additional fan activation is required. It shall be appreciated that, as compared to continuously operating the at least one fan, the periodic activation of the at least one fan under low hydrogen conditions consumes less power.

Having described various aspects of sensors for gas management of plurality of metal-air batteries, attention is now directed to certain aspects of drawing samples of gas from the shared vent 126 such that the first gas sensor 136A and/or the second gas sensor 136B may monitor concentration of a sensed gas (e.g., hydrogen) in the shared vent 126. In the description that follows, gas sampling from the shared vent 126 is described in the context of the first gas sensor 136A. It shall be appreciated that this is for the sake of clear and efficient description and, unless otherwise specified or made clear from the context, gas sampling for the second gas sensor 136B may be carried out according to the analogous techniques described below with respect to the first gas sensor 136A. Further, while the first gas sensor 136A and the second gas sensor 136B are schematically shown as positioned in the shared vent 156 in FIG. 1B, it shall be understood that this is for the sake of clarity of illustration. Unless otherwise, specified the first gas sensor 136A and/or the second gas sensor 136B may be mounted in fluid communication with the shared vent 156 via the assembly 301 (FIG. 3A), as described in the following paragraphs.

Referring now to FIGS. 1A-1C, FIGS. 3A-3J, and FIG. 4, the assembly 301 may include an adapter 302 and the first gas sensor 136A. The adapter 302 may be mounted to the ducting 125 such that the first gas sensor 136A may receive samples of gas from the shared vent 126 to determine concentration of one or more monitored gases (e.g., hydrogen). In general, to facilitate use of off-the-shelf components that are ubiquitous and cost-effectively sourced, the first gas sensor 136A may require a low volumetric flow rate (5 L/min) for accurate measurement of one or more components of gas flowing in the shared vent 126. By comparison, it may be useful to have a much higher flow rate (28.3 kL/min to 566 kL/min) in the shared vent 126 for safety reasons-namely, to reduce the likelihood of accumulation of hydrogen or another unwanted gas in the shared vent 126. As described in greater detail below, the adapter 302 may facilitate accommodating the design tension between the requirements associated with the low volumetric flow rate facilitating accurate gas concentration measurement by the first gas sensor 136A and the requirements associated with the much higher flow rate in the shared vent 126 for safe operation of the plurality of electrochemical cells 101.

In certain implementations, the adapter 302 of the assembly 301 may include a body 303, a first tube 304, and a second tube 306. The body 303 may define an opening 308 and a passage 310. The first tube 304 and the second tube 306 may each be supported on the body 303. The first tube 304 may define a first channel 312 and one or more first apertures 314. In certain implementations, the one or more first apertures 314 may be in fluid communication with the passage 310 via the first channel 312. The second tube 306 may define a second channel 316 and one or more second apertures 318. In some implementations, the one or more second apertures 318 may be in fluid communication with the passage 310 via the second channel 316. The body 303 may be mechanically coupled (for example, by a threaded coupler) to the ducting 125 with the first tube 304 and the second tube 306 each extending into the shared vent 126. The first gas sensor 136A may be mechanically coupled to (e.g., in threaded engagement with) the body 303 with the first gas sensor 136A disposed outside of the shared vent 126, as may be useful for facilitating electrical communication (e.g., wired and/or wireless) between the controller 133 and the first gas sensor 136A and/or for maintaining the first gas sensor 136A outside of the high-flow environment of the shared vent 126. The first apertures 314 may be disposed on an inlet side of the fan 130 in the shared vent 126. With the first gas sensor 136A mechanically coupled to the adapter 302 mounted to the ducting 125, a flow path 411 may be defined between the shared vent 126 and the first gas sensor 136A. That is, collectively, the one or more first apertures 314, the first channel 312, the passage 310, the second channel 316, and the one or more second apertures 318 may define at least a portion of a flow path 411 (shown in FIG. 4) of gas moving from the shared vent 156, through the first tube 304, to the first gas sensor 136A mounted to the body 303, and then returned to the shared vent 156 via the second tube 306.

In certain implementations, an outer surface 342 of the body 303 may be threaded to facilitate secure engagement with the ducting 125 of the system 100. For example, the outer surface 342 may be threaded according to standard threading specifications, such as ¾ inch National Pipe Tapered (NPT) threads, as may be useful for compatibility with commonly used ventilation components. Threading the outer surface 342 may facilitate installation into the ducting by screwing the body 303 into a corresponding threaded port in the ducting 125. The secure engagement provided by the threads may reduce the likelihood of leakage of gas or other contaminants around the adapter 302 while maintaining a stable mechanical connection, even under high-flow conditions that may be encountered in the shared vent 126 of the system 100. Further, or instead, the likelihood of leakage may be limited by the use of one or more sealing materials at an interface between the body 303 and the ducting 125.

The outer surface 342 may also facilitate modularity and ease of maintenance. For example, the outer surface 342 with standardized threading may facilitate quick removal and of the adapter 302 for maintenance and/or replacement. This may be useful for, among other thing, convenient sensor calibration or replacement without requiring substantial disassembly of the ducting 125.

In certain implementations, the one or more first apertures 314 defined by the first tube 304 may face in an opposite direction from the one or more second apertures 318 defined by the second tube 306. This opposing orientation of the one or more first apertures 314 relative to the one or more second apertures 318 may be useful for controlling flow of gas through the adapter 302. That is, with the one or more first apertures 314 arranged in the shared vent 126 to serve as an inlet into the first channel 312, the one or more second apertures 318 are an outlet from the second channel 316. The opposing orientation of the one or more first apertures 314 relative to the one or more second apertures 318 may reduce the likelihood that effluent from the one or more second apertures 318 may become inadvertently entrained again into the one or more first apertures 314. Gas entering through the one or more first apertures 314 moves through the first channel 312 and is directed through the passage 310 defined within the body 303 of the adapter 302. The gas in the passage 310 has a flow rate (e.g., 5 L/min or less) compatible with the first gas sensor 136A before exiting via the second channel 316 and the second apertures 318. Thus, opposing arrangement of the one or more first apertures 314 relative to the one or more second apertures 318 may contribute to effective airflow management, reducing the likelihood of interference from turbulence or backflow that might affect performance of the first gas sensor 136A. For example, the opposing arrangement of the one or more first apertures relative to the one or more second apertures 318 may facilitate directing a consistent sample of gas from the ducting 125 to the first gas sensor 136A, even in high-flow environments. This configuration may be particularly advantageous for systems requiring a combination of precision and speed in measuring changes in hydrogen concentration in the shared vent 126 because the sample of the gas entering the first gas sensor 136A is representative of the bulk gas flow moving through the ducting 125 at any particular time. Stated differently, the reduction in inadvertent entrainment of backflow from the one or more second apertures 318 into the one or more first apertures 314 may, among other things, improve the temporal response of gas (e.g., hydrogen) concentration measurements made by the first gas sensor 136A, which may facilitate initiating one or more corrective safety measures before an adverse event (e.g., ignition of an oxyhydrogen mixture) has time to occur in the shared vent 126 of the ducting 125.

In certain instances, the body 303 may include a threaded portion 340 along the passage 310 to facilitate releasable engagement with the first gas sensor 136A. For example, the threads of the threaded portion 340 of the body 303 along the passage 310 may align with the corresponding threads on the first gas sensor 136 to form a secure mechanical coupling of the first gas sensor 136 to the body 303 while contributing to easy and intuitive installation and/or removal of the first gas sensor 136A. This threaded engagement may useful, for example, for maintaining the first gas sensor 136A firmly positioned within the passage 310, even under varying flow conditions within the passage 310, while maintaining a reliable seal between the first gas sensor 136A and the body 303 to reduce the likelihood of gas leaks from the shared vent 126 or contamination of the shared vent 126. The threads may be precision-engineered for compatibility with industry-standard gas sensors, such as those designed for hydrogen detection.

The threaded portion 340 may be disposed within the passage 310 such that the first gas sensor 136A is properly positioned relative to the flow path 411. For example, with the first gas sensor 136A fully engaged with the threaded portion 340 of the body 303 within the passage 310, a sensing element of the first gas sensor 136A may be positioned directly in the flow path 411, as may be useful for making accurate measurements of gas concentration in the flow path 411. Further, or instead, the threaded portion 340 may contribute to the modularity of the system, facilitating quick replacement or maintenance of the first gas sensor 136A with little disruption to the overall operation of the system 100. This feature may be particularly advantageous in safety-critical applications, such as detecting hydrogen concentrations in metal-air battery systems, where sensor accuracy and secure engagement may be critical aspects of performance of the system 100.

In general, the opening 308 may provide access to the passage 310. In turn, the passage 310 may serves as a conduit for a low volumetric flow (e.g., 5 L/min) of gas into proximity with the first gas sensor 136A such that concentration of one or more gas components may be measured by the first gas sensor 136A, which may be calibrated for operation within the low volumetric flow of gas moving along the flow path 411. As an example, the first gas sensor 136A may be mounted within the opening 308 such that the first gas sensor 136A extends into the passage 310. With this alignment, gas entering the adapter 302 from the first apertures 314 and traveling through the first channel 312 may flow directly through the passage 310, where the gas flowing through the adapter 302 may interact with one or more portions (e.g., a sensing element) of the first gas sensor 136A. As the gas moves along the flow path 411, the exit path from the passage 310 leads to the second channel 316 and the second apertures 318 and back into the shared vent 126.

The passage 310 may be centrally located within the body 303, as may be useful for protecting the first gas sensor 136A disposed in the body 303 and provides symmetry that may be less likely to result in variations in performance of the adapter 302. Further, or instead, the opening 308 directly above the passage 310 may facilitate easy installation and removal of the first gas sensor 136A while maintaining a secure, sealed interface while the first gas sensor 136A is mechanically coupled to the adapter 302.

In general, the one or more first apertures 314 may act as an inlet for gas, moving along the flow path 411, into the first channel 312. The first tube 304 may be aligned relative to the body 303 such that gas entering through the first apertures 314 and moving along the first channel 312 may be directed efficiently (e.g., without introducing large amounts of turbulence) into the passage 310, where the gas may be measured by the first gas sensor 136A.

In general, the second tube 306 extends from the body 303 in an orientation in which the second channel 216 is parallel to the first channel 312. The one or more second apertures 318 defined by the second tube 306 may serve as outlets for gas, moving along the flow path 411) and exiting the adapter 302 for reintroduction into the shared vent 126. As discussed above, the separation between the one or more first aperture 314 and the one or more second aperture 318 may reduce the likelihood of inadvertent backflow of gas into he adapter 302.

In certain instances, the first tube 304 may have a first closed end 320 that terminates away from the body 303 and, further or instead, the second tube 306 may have a second closed end 322 that terminates away from the body 303. The first closed end 320 and the second closed end 322 may be sealed such that gas flow through the adapter 302 is exclusively through the one or more first apertures 314 and the one or more second apertures 318, thus controlling the direction of the flow path 411 for ingress into and egress from the adapter 302. That is, the first closed end 320 and the second closed end 322 may reduce the likelihood of gas bypassing the one or more first apertures 314 such that sampled gas enters through the first apertures 314, flows through the first channel 312, the passage 310, and the second channel 316, and exits through the second apertures 318. Further, or instead, the change in direction (e.g., by about 90 degrees) required for gas to flow into the one or more first apertures 314 and move along the first channel 312 may facilitate controlling the volumetric flow rate of gas incident on the first gas sensor 136A. Still further, or instead, the change in direction required for gas to flow into the one or more first apertures 314 and move along the first channel 312 may facilitate removing debris from the gas moving along the flow path 411, thus protecting the first gas sensor 136A from potential damage or contamination that may otherwise result from exposure to such debris.

As compared to tubes having open ends, the first closed end 320 of the first tube 304 and the second closed end 322 of the second tube 306 may contribute to the structural integrity of the adapter 302. That is, the first closed end 320 of the first tube 304 and the second closed end 322 of the second tube 306 may provide rigidity to the first tube 304 and the second tube 306, respectively. In environments in which the adapter 302 is exposed to high volumetric flow rates of gas moving through the shared vent 126 and/or mechanical vibrations from the system 100 (e.g., during operation, installation, or transport), the first closed end 320 and the second closed end 322 may help maintain integrity of the first channel 312 and the second channel 316, respectively. In some implementations, the first closed end 320 and/or the second closed end 322 may be fabricated from the same material as the first tube 304 and the second tube 306, as may be useful for cost-effective fabrication, compatibility with the gases in the system, or resistance to corrosion or wear, or a combination thereof.

In some implementations, the first tube 304 and the second tube 306 may have equal lengths extending from the body 303. This symmetry may be useful, for example, for reducing the likelihood of inadvertent turbulence and/or recirculation zones that may adversely impact the intended direction of the flow path 411. Equal lengths of the first tube 304 and the second tube 306 may further, or instead, facilitate cost-effective manufacturing and assembly of the adapter 302.

In some implementations, the first channel 312 of the first tube 304 and the second channel 316 of the second tube 306 may have equal (to within manufacturing tolerances) minimum cross-sectional areas transverse to their respective length dimensions. This may be useful, for example, so that the airflow through the first channel 312 and the second channel 316 may be subject to the same flow resistance and velocity constraints, facilitating a balanced flow path through the adapter 302 with a reduced likelihood of unintended gas recirculation within the adapter 302. With equal minimum cross-sectional areas, the first tube 304 and the second tube 306 may promote uniform gas sampling, reducing the likelihood of differential pressure or turbulence that could disrupt the flow and compromise the accuracy of the gas sensor 324. Further, or instead, equal minimum cross-sectional areas of the first channel 312 and the second channel 316 may facilitate cost-effective manufacturing of the adapter 302.

In general, the one or more first apertures 314 may be defined by the first tube 304 such that gas in the shared vent 126 may enter the first channel 312 via the one or more first apertures 314 along the flow path 411. In instances in which the one or more first apertures 314 include a plurality of instances of the first aperture 314, the plurality of instances of the first aperture 314 may be uniformly spaced relative to one another along a length of the first tube 304, as may be useful for receiving a representative gas sample into the adapter 302 from the high volumetric gas flow in the shared vent 126. Further, or instead, in instances in which the one or more first apertures include a plurality of instances of the first aperture 314, a diameter 330 may be uniform across all instances of the plurality of the first apertures 314 as may be useful for promoting consistent flow characteristics for the gas entering the first channel 312 and ease of manufacturing. This uniformity may be useful, for example, for promoting a predictable and stable flow of gas through the adapter 302, reducing the likelihood of introducing unintended variation between the gas flow in the shared vent 126 and the gas moving through the adapter 302.

In general, the one or more second aperture 318 may be defined by the second tube 306 such that gas moving along the flow path 411 in the second channel 316 may reenter the shared vent 126 via the one or more second apertures 318. In in stances in which the one or more second apertures 318 includes a plurality of instances of the second aperture 318, each instance of the second aperture may have a the same (to within manufacturing tolerances) diameter 332, as may be useful for reintroducing the gas into the shared vent 126 with a reduced likelihood of creating unintended backflow into the one or more first apertures 314.

The diameter 330 the one or more first apertures 314 and the diameter 332 of the one or more second apertures 318 may be 1 mm to 3 mm (e.g., about 2 mm). Apertures within this size range may facilitate achieving a balance between restricting the flow rate of the gas moving along the flow path 411 for compatibility with the first gas sensor 136A (for operation within calibration range) while maintaining sufficient throughput for measurements that may rapidly detect changes in concentration of hydrogen or other gas component in the shared vent 126.

In some implementations, the one or more first apertures 314 collectively have a first open area, while the one or more second apertures 318 may collectively have a second open area that is smaller than the first. This difference in open areas may create a flow restriction that helps control the volumetric flow rate of the gas passing through the adapter 302 along the flow path 411. The difference in open areas may be achieved, for example, by having fewer instances of the second aperture 318 relative to instances of the first aperture 314, by forming each instance of the second aperture 318 with a smaller diameter than the that of each instance of the first aperture 314, or by a combination of smaller and/or fewer apertures.

In some implementations, the ratio of the first open area of the one or more first apertures 314 to the second open area of the one or more second apertures 318 may be greater than 2:1 and less than 5:1. For example, the ratio may be approximately 3:1. This ratio may balance the need for temporally responsive sensing with the operational requirements of the first gas sensor 136A.

In certain implementations, the body 303 may define a first through hole 350 and a second through hole 352. The first through hole 350 and the second through hole 352 may provide structural support for the first tube 304 and the second tube 306, respectively. The through holes 350, 352 may be machined to align with the intended positioning of the first tube 304 and the second tube 306, respectively, relative to one another and relative to the body 303. The first through hole 350 may accommodate the first tube 304, which defines the first channel 312 and contains the one or more first apertures 314. Similarly, the second through hole 352 may accommodate the second tube 306, which defines the second channel 316 and houses the one or more second apertures 318. As an example, the first tube 304 may be brazed or welded into the first through hole 350, and the second tube 306 may be brazed or welded into the second through hole 352. These attachment techniques may create a durable and gas-tight seal between the body 303 and each of first tube 304 and the second tube 306, reducing the likelihood of an unintended leak path for gas to escape the adapter 302 while also providing structural integrity to the first tube 304 and the second tube 306. Brazing or welding provide a strong and permanent connection, which may be useful for withstanding the mechanical stresses and thermal fluctuations that may occur in energy storage applications.

In general, the volumetric flow rate of gas along the flow path 411 in the adapter 302 may be between 0.001% and 0.01% of the bulk volumetric flow rate in the shared vent 126 (e.g., the volumetric flow rate initially incident on the first tube 304 along the one or more instances of the first apertures 314). This substantial reduction may be achieved through any one or more of the various techniques described herein (e.g., number and sizing of apertures, sizing of flow restrictions, etc.). The reduced flow rate in the adapter 302 may increase the likelihood that gas entering the first gas sensor 136A may be consistently within a rated operating range of the first gas sensor 136A.

In some implementations, the volumetric flow rate at the first gas sensor 136A, at least partially disposed within the passage 310 of the adapter 302, may be passively controlled (e.g., by sizing and/or shape) by the adapter 302 to remain below 5 L/min, even when the fan 130 in the shared vent 126 operates at a much higher volumetric flow rate, typically ranging from 28.3 kL/min to 566 kL/min.

Additionally, or alternatively, while systems for gas management have been described as venting oxyhydrogen mixtures, it shall be appreciated that other approaches are additionally or alternatively possible. For example, oxygen and hydrogen may be vented separately from each one of a plurality of electrochemical cells. With oxygen and hydrogen vented separately, the likelihood of an ignitable mixture of hydrogen and oxygen/air decreases. However, such an arrangement requires additional hardware, as compared to techniques described herein for venting oxyhydrogen mixtures. For example, to vent oxygen and hydrogen separately, each one of the electrochemical cells may include a separator disposed between at least one air electrode and a metal electrode. The separator may divide the headspace into a first portion and a second portion fluidically isolated from one another. The first portion of the headspace may be above the metal electrode and in fluid communication with the shared vent of the ducting. The second portion of the headspace may be above the at least one air electrode and in fluid communication with an oxygen vent exhausting to an ambient atmosphere.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. At the same time, processing may be distributed across devices such as the various systems described above, or all of the functionality may be integrated into a dedicated, standalone device. All such permutations and combinations are intended to fall within the scope of the present disclosure.

Embodiments disclosed herein may include computer program products comprising computer-executable code or computer-usable code that, when executing on one or more computing devices, performs any and/or all of the steps of the control systems described above. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the control systems described above may be embodied in any suitable transmission or propagation medium carrying computer-executable code and/or any inputs or outputs from the same.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the scope of the disclosure.

What is claimed is:

1. An assembly for sensing gas concentration in a ventilation system, the assembly comprising:
a body defining an opening and a passage;
a first tube supported on the body, the first tube defining a first channel and one or more first apertures, the one or more first apertures in fluid communication with the passage via the first channel; and
a second tube supported on the body, the second tube defining a second channel and one or more second apertures, the one or more second apertures in fluid communication with the passage via the second channel and, collectively, the one or more first apertures, the first channel, the passage, the second channel, and the one or more second apertures defining at least a portion of a flow path.

2. The assembly of claim 1, wherein the one or more first apertures collectively have a first open area, and the one or more second apertures collectively have a second open area less than the first open area.

3. The assembly of claim 2, wherein a ratio of the first open area to the second open area is greater than 2:1 and less than 5:1.

4. The assembly of claim 3, wherein the ratio of the first open area to the second open area is 3:1.

5. The assembly of claim 2, wherein each of the one or more first apertures and each of the one or more second apertures has a diameter of 1 mm to 3 mm.

6. The assembly of claim 5, wherein each of the one or more first apertures and each of the one or more second apertures has a diameter of 2 mm.

7. The assembly of claim 1, wherein at least a portion of the passage is threaded such that the passage is releasably connectable in threaded engagement with a gas sensor.

8. The assembly of claim 1, wherein an outer surface of the body is threaded such that the body is threaded engagement with ducting of the ventilation system.

9. The assembly of claim 1, wherein the body defines a first through hole and a second through hole, the first tube is supported in the first through hole, and the second tube is supported in the second through hole.

10. The assembly of claim 9, wherein the first tube is brazed or welded in the first through hole, and the second tube is brazed or welded in the second through hole.

11. The assembly of claim 1, wherein the first tube has a first minimum cross-sectional area transverse to a length dimension of the first tube, the second tube has a second minimum cross-sectional area transverse to a length dimension of the second tube, and the first minimum cross-sectional area is equal to the second minimum cross-sectional area.

12. The assembly of claim 1, wherein at least one of the first tube or the second tube has a closed end away from the body.

13. The assembly of claim 1, further comprising a gas sensor disposed in the passage via the opening of the body.

14. A ventilation system for electrochemical energy storage and discharge, the ventilation system comprising:
ducting defining a shared vent connectable in fluid communication with a plurality of electrochemical cells;
at least one fan in fluid communication with the shared vent;
an adapter including a body, a first tube, and a second tube, the body defining an opening and a passage in fluid communication with one another, the body mechanically coupled to the ducting with the first tube and the second tube in the shared vent; and
a gas sensor in fluid communication with the passage, and the passage in fluid communication with the shared vent via the first tube and the second tube.

15. The ventilation system of claim 14, wherein the first tube defines one or more first apertures, the second tube defines one or more second apertures, and the one or more first apertures and the one or more second apertures are disposed on an inlet side of the at least one fan.

16. The ventilation system of claim 14, wherein the body of the gas sensor is in threaded engagement with the ducting.

17. The ventilation system of claim 14, wherein the gas sensor is in threaded engagement with the body of the adapter.

18. The ventilation system of claim 14, wherein the gas sensor has a rated volumetric flow rate less than 5 L/min, and the at least one fan has a volumetric flow rate is 28.3 kL/min to 566 kL/min.

19. The ventilation system of claim 14, wherein volumetric flow rate at the gas sensor is less than 5/L/min at a constant absolute pressure of 1.0±0.2 atm.

20. The ventilation system of claim 14, wherein the gas sensor is a hydrogen sensor.
